(19) United States
(12) Patent Application Publication
(10) Pub. No.: US 2006/0149301 A1
(43) Pub. Date: Jul. 6, 2006

(54) Phacoemulsification system utilizing graphical user interfaces for adjusting pulse parameters

(76) Inventor: Michael J. Claus, Newport Coast, CA (US)

Correspondence Address: Advanced Medical Optics, Inc.;Legal Department, 1700 E. St. Andrew Place, Santa Ana, CA, 92705, US

(21) Appl. No.: 11/030,443

(22) Filed: Jan. 5, 2005

(51) Int. Cl. A61B 17/32

(52) U.S. Cl. 606/169

(57) ABSTRACT

A phacoemulsification system with enhanced user utility and tactile operability includes a control unit and a handpiece with a needle and a vibrating unit that is configured to ultrasonically vibrate the needle. The handpiece includes a needle and a vibrating unit for ultrasonically vibrating the needle according to a variable parameter, such as frequency, shape, size, duty cycle, and so on. The control unit is configured to adjust the value of the parameter. A monitor is operably connected to the control unit for displaying a graphical user interface (GUI). The GUI includes an adjustable control element, such as a slider bar, radio buttons, or handles, and an indication element that indicates the value of the parameter, either numerically or graphically. An interface device, such as a touchscreen, a mouse, or a keyboard, is operably connected to the control unit for enabling a user to adjust the control element. The control unit is configured to change the value of the parameter at least in response to adjustments of the control element by the user.

### BACKGROUND OF THE INVENTION

The invention relates to methods and devices for removing a lens from an eye and, more particularly, phacoemulsification systems.

A number of medically recognized techniques have been utilized for removing a lens from an eye. One such technique is phacoemulsification, which includes making a corneal incision in the eye and then inserting a needle of a handpiece into the eye, with the needle being ultrasonically driven or vibrated in order to emulsify the eye lens. Simultaneously with this emulsification, a fluid is utilized for irrigating the eye, and a vacuum is provided for aspirating the emulsified lens and inserted fluids.

Pulses or signals from a control unit controls the vibration of the needle. The pulses are controlled by one or more parameters, such as frequency, size, shape, and so on. It is inconvenient during a phacoemulsification procedure to change the parameters of the pulses being delivered to the needle in real time without the surgeon being distracted during the change.

In view of the foregoing, there remains a need in the art for a phacoemulsification system that has enhanced user utility and tactile operability so that parameters of the pulses can be changed in real time during a procedure.

### SUMMARY OF THE INVENTION

According to one aspect of the invention, a phacoemulsification system with enhanced user utility and tactile operability includes a control unit and a handpiece with a needle and a vibrating unit that is configured to ultrasonically vibrate the needle. The handpiece includes a needle and a vibrating unit for ultrasonically vibrating the needle according to at least one variable parameter, such as frequency, shape, size, duty cycle, amplitude, and so on. The control unit is configured to adjust the value of the parameter that is varied. In a number of embodiments, multiple parameters may be adjusted.

The system also includes a monitor operably connected to the control unit for displaying a graphical user interface (GUI). The GUI includes an adjustable control element, such as a slider bar, radio buttons, or handles, and an indication element that indicates the value of the parameter, either numerically or graphically. An interface device, such as a touchscreen, a mouse, or a keyboard, is operably connected to the control unit for enabling a user to adjust the control element. The control unit is configured to change the value of the parameter at least in response to adjustments of the control element by the user.

One of the advantages of the invention is that during a phacoemulsification procedure, a surgeon or surgical assistant can view the GUI on the monitor to easily see parameters of the pulse currently being provided to the handpiece. In addition, the surgeon or surgical assistant can easily and tactilely adjust the value of one or more parameters on the same GUI. This greatly increases the utility of the system during a highly refined and sensitive procedure. As mentioned above, in many cases, the values of multiple parameters can be adjusted.

Other features and advantages of the present invention will become apparent to those skilled in the art from a consideration of the following detailed description taken in conjunction with the accompanying drawings.

### BRIEF DESCRIPTION OF THE SEVERAL VIEWS OF THE DRAWINGS

FIG. 1 is a block diagram of a phacoemulsification system;

FIG. 2 schematically illustrates a handpiece of the system in relation to an eye;

FIG. 3 illustrates a commercial embodiment of the invention;

FIG. 4 is a block diagram of a computer and a monitor of the system;

FIG. 5 illustrates a graphical user interface (GUI) according to a number of embodiments;

FIG. 6 illustrates a touchscreen interface device;

FIG. 7 illustrates a mouse interface device;

FIG. 8 illustrates a keyboard interface device;

FIG. 9 illustrates a GUI according to other embodiments;

FIG. 10 illustrates a GUI according to still other embodiments;

FIG. 11 illustrates a GUI according to further embodiments;

FIG. 12 illustrates a footswitch interface device; and

FIG. 13 illustrates a remote-control interface device.

### DETAILED DESCRIPTION OF THE INVENTION

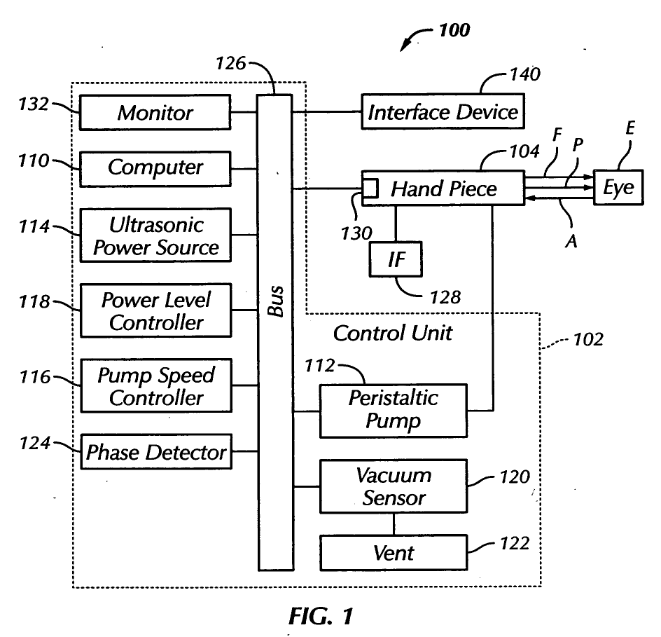

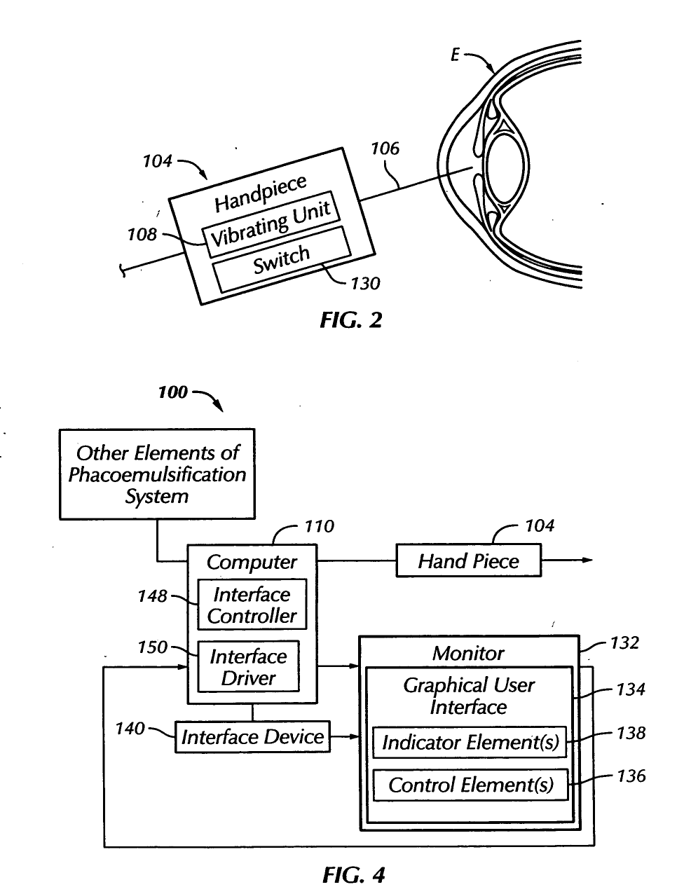

Referring to the drawings in more detail, a phacoemulsification system 100 with enhanced user utility and tactile operability is illustrated in FIG. 1. In a number of embodiments, the system 100 may include a control unit 102 and a handpiece 104 operably coupled together. As shown in FIG. 2, the handpiece 104 may include a needle 106 for insertion into an eye E and a vibrating unit 108 that is configured to ultrasonically vibrating needle 106. The vibrating unit 108, which may include, e.g., a piezoelectric crystal, vibrates the needle 106 according to one or more parameters, such as frequency, pulse width, shape, size, duty cycle, amplitude, and so on, which is discussed in more detail below. The system 100 is described in general immediately hereunder, with a detailed description of the enhanced user utility and operability of the system following.

With particular reference to FIG. 1, the functional block diagram of the phacoemulsification system 100 includes a microprocessor computer 110 which is operably connected to and controls the various other elements of the system. In a number of embodiments, the system 100 may include a variable speed pump 112 for providing a vacuum source and a pulsed ultrasonic power source 114 for providing control outputs to a pump speed controller 116 and an ultrasonic power level controller 118. A vacuum sensor 120 provides an input to the computer 110 representing the vacuum level on the output side of the pump 112. Venting may be provided by a vent 122. The system 100 may also include a phase detector 124 for providing an input to the computer 100 that represents a phase shift between a sine wave representation of the voltage applied to the handpiece 104 and the resultant current into the handpiece 104. The functional representation of the system 100 also includes a system bus 126 for enable the various elements to be operably coupled with each other.

In operation, the control unit 102 supplies ultrasonic power on to the phacoemulsification handpiece 104. An irrigation fluid source 128 provides irrigation fluid to the handpiece 104. The irrigation fluid and an ultrasonic pulse are applied by the handpiece 104 to a patient's eye E, which are indicated by arrows F and P. Aspiration of the eye E is achieved by means of the pump 112, which is indicated by arrow A. The handpiece 104 may include a switch 130 for enabling a surgeon to select an amplitude of electrical pulses to the handpiece 104 via the computer 110, the power level controller 118, and the ultrasonic power source 114. The operation of the system 100 in general may be in accordance with the disclosure of U.S. Pat. No. 6,629,948, which is incorporated herein in its entirety by reference.

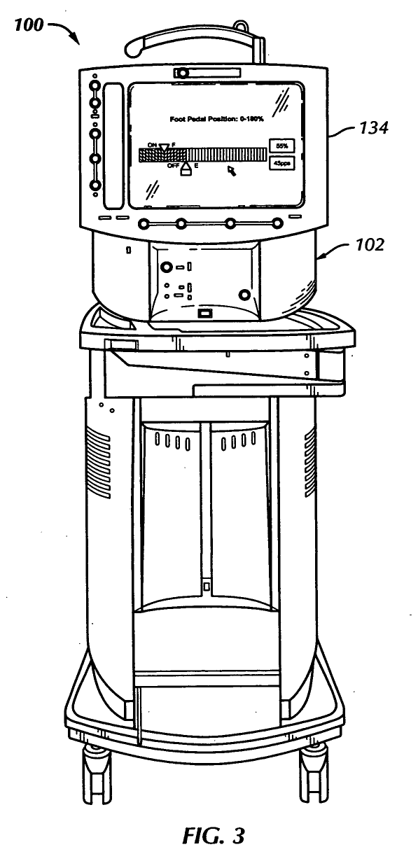

Turning to the enhanced user utility and operability of the system 100 in more detail, the control unit 102 may include a video display or monitor 132 as shown in FIGS. 3 and 4. The monitor 132 is operably connected to the computer 110 and is configured to display a graphical user interface (GUI) 134. An example of a GUI 134 is shown in FIG. 4. The GUI 134 may include one or more adjustable control elements 136 and one or more indication elements 138. The indication elements 138 either graphically or numerically indicate the value of the operating parameters of the handpiece 104 or the irrigation fluid. For example, as shown in FIG. 5, indication element 138a indicates a duty cycle of the pulse, and indication element 138b indicates the pulse rate of the pulse.

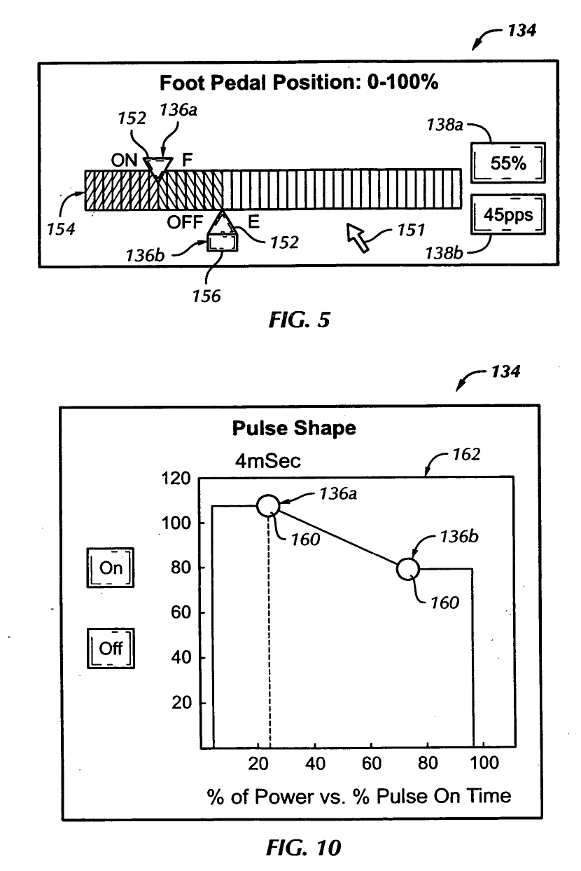

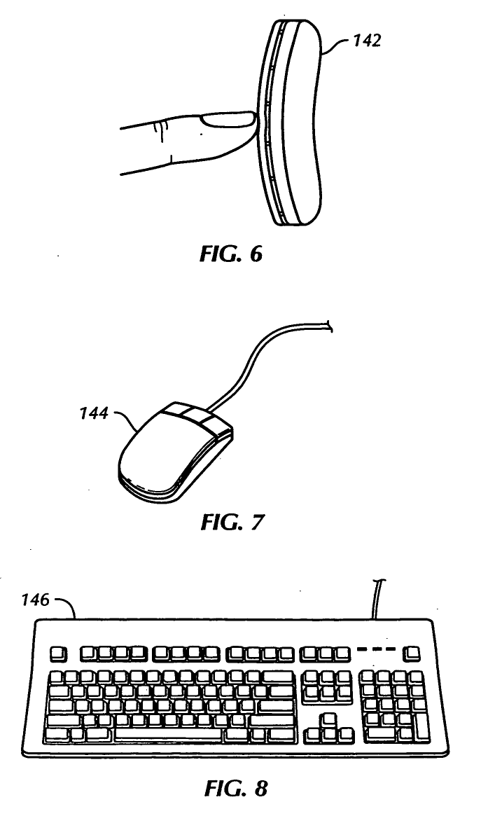

As shown in FIG. 4, the system 100 may also include an interface device 140 operably connected to the control unit 102 (as shown in FIG. 1) or the computer 110. The interface device 140 is configured to enable a user to adjust the control element 136 and, therefore, to change the value of the parameter or parameters of the handpiece 104. The control unit 102 is configured to change the value of the parameter at least in response to adjustments of the control element 136 by the user. For example, the interface device 140 may include a touchscreen 142 as shown in FIG. 6 integrated with the monitor 132 for tactile or manual adjustment of the control elements 136, a mouse 144 as shown in FIG. 7 for curser adjustment of the control elements 136, or a keypad or keyboard 146 as shown in FIG. 8 for keystroke adjustment of the control elements 136. The computer 110 may include an interface controller 148 and an interface driver 150 for controlling the interface device 140. In mouse 144 embodiments, the GUI 134 may display a pointer 151 operatively associated with the mouse 144.

In a number of embodiments, the computer 110 may be configured to change the values of the parameters in real time in response to any change made with the control elements 136, with the indication elements 138 also indicating the new values in real time. This enables the user to quickly control and modify the pulses P at the handpiece 104 in response to desired changes during a phacoemulsification procedure. The changes in the parameter values correspondingly result in changes to the pulses P provided by the handpiece 104.

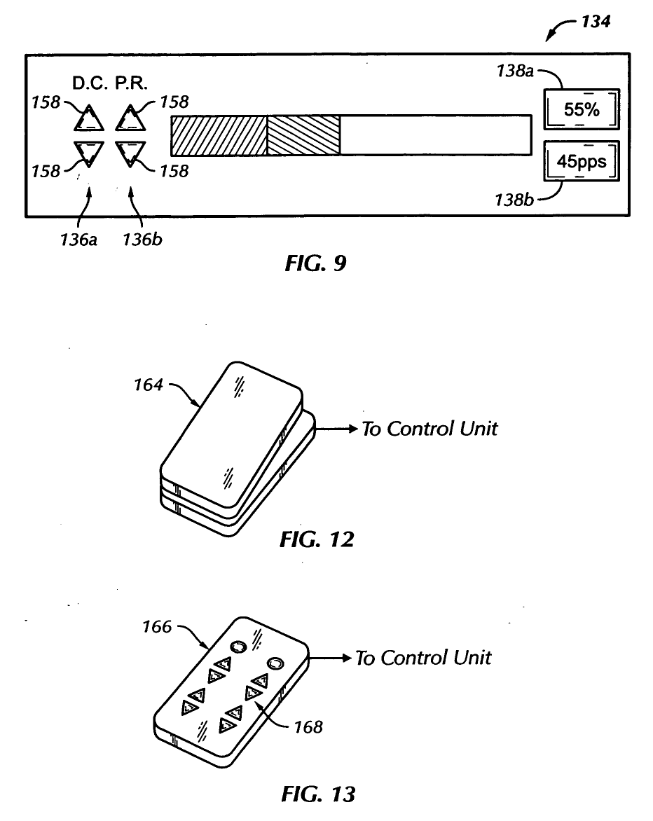

In some of the embodiments as shown in FIG. 5, each of the control elements 136 of the GUI 134 may include a slider bar 152 juxtaposed on a scale 154. One of the slider bars 152 may include a common tab 156 which when actuated moves both of the slider bars 152 simultaneously. In other embodiments, such as shown in FIG. 9, each of the control elements 136 may include a set of radio buttons 158 which may be actuated by means of the interface device 140 to increase and decrease the value of a respective parameter. In addition to a horizontal representation of the parameter values, vertical or circular representations may be used.

In addition to changing parameter values, the GUI 134 may also be utilized to change the shape of the pulse P. For example, as shown in FIG. 10, each of the control elements 136 may include a handle 160 juxtaposed over a graph 162 that, for example, represents the shape of the pulse P in terms of percentage power versus percentage of pulse ON time. A user may manipulate the position of the handles 160 with the interface device 140 to change the shape of the pulse P in real time.

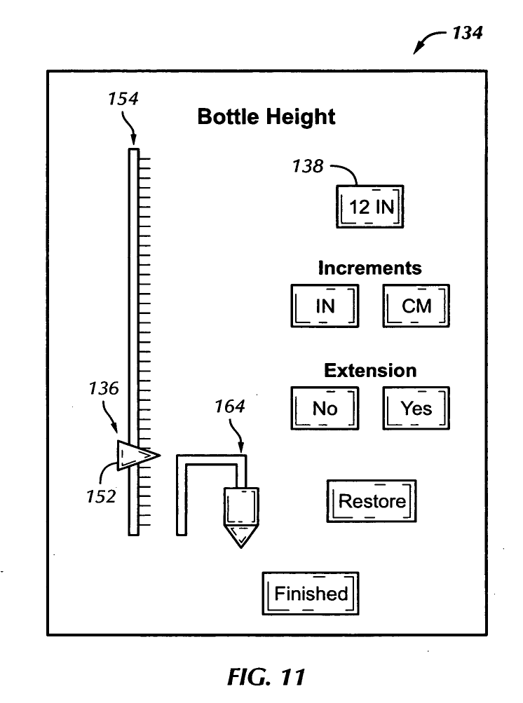

In addition to displaying a GUI 134 for changing parameter values, the monitor 132 may also display a GUI 134 that can be utilized for changing the height of a bottle of irrigation fluid 128 mounted on an IV pole or for changing the pressure by which irrigation fluid is infused into the eye. More specifically, as shown in FIG. 11, in these embodiments, the control element 136 may include a slider bar 152 juxtaposed on a scale 154. The GUI 134 may also include an icon 164 of the bottle of fluid 128 that is configured to move correspondingly with the slider bar 152 so that the scale 154 graphically represents the current height of the bottle. An indication element 138 may also numerically indicate the height as well.

With reference to FIG. 2, in use the needle 106 may be operatively inserted into an eye E. The user may then cause the control unit to provide the pulse P to the handpiece 104, thereby causing the needle 106 to vibrate. One or more of the control element 136 may then be manipulated to manually change the value of respective parameters of the pulse P. In touchscreen 142 embodiments, the control element 136 may be moved or actuated by a user pressing a finger against the surface of the monitor 132 over the graphical representation of the control element 136, and then dragging, sliding, or moving the finger across the surface. In mouse 144 embodiments, the control element 136 may be moved by placing the pointer 151 on the graphical representation of the control element 136, and then moving the element as desired. In keyboard 146 embodiments, designated keys may be associated with a parameter value so that by manipulating the keys, the value of the parameter changes.

Those skilled in the art will understand that the preceding exemplary embodiments of the present invention provide the foundation for numerous alternatives and modifications thereto. For example, in addition to the examples provided above, the interface device 140 may also a footswitch 164 as shown in FIG. 12 that is operably connected to the control unit 102. A surgeon or surgical assistant can operate the footswitch 164 to provide inputs to the control unit 102 to, e.g., adjust the values of the parameters or change the irrigation fluid flow, as desired. In addition, the interface device 140 may also include a remote control 166 as shown in FIG. 13 that may either directly or wirelessly communicate with the control unit 102. The remote control 166 may include a plurality of buttons 168 that correspond to the control elements 136 of the GUI 134 for surgical control of the parameters. These and other modifications are also within the scope of the present invention. Accordingly, the present invention is not limited to that precisely as shown and described above but by the scope of the appended claims.

What is claimed is:

1. An apparatus comprising:
a handpiece including a needle and a vibrating unit for ultrasonically vibrating the needle according to a variable parameter;
a control unit operably coupled to the handpiece for adjusting the parameter;
a monitor operably connected to the control unit for displaying a graphical user interface (GUI), the GUI including an adjustable control element and an indication element, the indication element for indicating the value of the parameter; and
an interface device operably connected to the control unit for enabling a user to adjust the control element;
the control unit being configured to change the value of the parameter at least in response to adjustments of the control element by the user.

2. The apparatus of claim 1 wherein the value of the parameter is changeable in real time.

3. The apparatus of claim 1 wherein interface device is adapted for manual adjustment of the control element by the user.

4. The apparatus of claim 1 wherein the vibrating unit further comprises a pulse that is controlled by the parameter.

5. The apparatus of claim 1 wherein the interface device includes a touchscreen operatively integrated with the monitor.

6. The apparatus of claim 1 wherein the interface device includes a mouse with an associated pointer integrated with the GUI.

7. The apparatus of claim 1 wherein the interface device includes a footswitch.

8. The apparatus of claim 1 wherein the interface device includes a remote control.

9. The apparatus of claim 1 wherein the control element includes a slider bar.

10. The apparatus of claim 1 wherein the control element includes radio buttons.

11. The apparatus of claim 1 wherein the control element includes a handle.

12. The apparatus of claim 1 wherein the parameter includes a frequency of the pulse.

13. The apparatus of claim 1 wherein the parameter includes a size of the pulse.

14. The apparatus of claim 1 wherein the parameter includes a shape of the pulse.

15. The apparatus of claim 1 wherein the parameter includes an amplitude of the pulse.

16. The apparatus of claim 1 wherein the control unit is configured to cause the GUI to display the value of the parameter being provided to the handpiece in real time.

17. The apparatus of claim 1 wherein the vibrating unit of the handpiece vibrates the needle according to a plurality of parameters;
the GUI including a corresponding plurality of control elements and indicator elements respectively associated with the plurality of parameters;
the interface device being configured to enable the user to manually adjust each of the control elements independently of each other; and
the control unit being configured to change the values of the parameters in response to adjustments of the control elements by the user and to provide the control signal to the vibrating unit of the handpiece with the changed values of the parameters in real time.

18. The apparatus of claim 1 wherein the GUI further includes an adjustable control element and an indication element associated with a height of a bottle of irrigation fluid mounted on an IV pole;
the control unit being configured to change the height of the bottle at least in response to adjustments of the control element associated therewith by the user.

19. A method for operating a phacoemulsification system, the method comprising:
providing a phacoemulsification system including:
a handpiece including a needle and a vibrating unit for ultrasonically vibrating the needle according to a variable parameter;
a control unit operably coupled to the handpiece for adjusting the parameter;
a monitor operably connected to the control unit for displaying a graphical user interface (GUI), the GUI including an adjustable control element and an indication element, the indication element for indicating the value of the parameter; and
an interface device operably connected to the control unit for enabling a user to adjust the control element;
the control unit being configured to change the value of the parameter at least in response to adjustments of the control element by the user;
placing the handpiece in an operative relationship with an eye;
causing the control unit to provide the control signal to the handpiece; and
manipulating the control element of the GUI manually to change the value of the parameter.

20. The method of claim 19 wherein the interface device includes a touchscreen operatively integrated with the monitor, the manipulating step comprising:
contacting the touchscreen at the control element with a finger.

21. The method of claim 19 wherein the interface device includes a mouse with an associated pointer integrated with the GUI, the manipulating step comprising:
positioning the pointer on the control element.

22. The method of claim 19 wherein the control element includes a slider bar, the manipulating step comprising:
moving the slider bar.

23. The method of claim 19 wherein the control element includes radio buttons, the manipulating step comprising:
actuating the radio buttons.

24. The method of claim 19 wherein the control element includes a handle, the manipulating step comprising:
moving the handle.

25. The method of claim 19 wherein the parameter includes a frequency of the pulse, the manipulating step comprising:
manipulating the control element to change the value of the frequency of the pulse.

26. The method of claim 19 wherein the parameter includes a size of the pulse, the manipulating step comprising:
manipulating the slider bar to change the size of the pulse.

27. The method of claim 19 wherein the parameter includes pulse shape, the manipulating step comprising:
manipulating the slider bar to change the shape of the pulse.

28. The method of claim 19 wherein the vibrating unit of the handpiece vibrates the needle according to a plurality of parameters, the GUI includes a corresponding plurality of control elements and indicator elements respectively associated with the plurality of parameters, the interface device is configured to enable the user to manually adjust each of the control elements independently of each other, and the control unit is configured to change the values of the parameters in response to manipulation of the control elements by the user and to provide the control signal to the vibrating unit of the handpiece with the changed values of the parameters in real time, the manipulating step comprising:
manipulating at least one of the control element of the GUI manually to change the value of a corresponding one of the parameters.

29. A phacoemulsification system including an handpiece with a needle and vibrating unit for ultrasonically vibrating the needle according to a variable parameter, the system comprising:
a control unit operably coupled to the handpiece for adjusting the parameter;
a monitor operably connected to the control unit for displaying a graphical user interface (GUI), the GUI including an adjustable control element and an indication element, the indication element for indicating the value of the parameter; and
an interface device operably connected to the control unit for enabling a user to adjust the control element;
the control unit being configured to change the value of the parameter at least in response to adjustments of the control element by the user.

30. The system of claim 29 wherein the interface device includes a touchscreen operatively integrated with the monitor.

31. The system of claim 29 wherein the interface device includes a mouse with an associated pointer integrated with the GUI.

32. The system of claim 29 wherein the control element includes a slider bar.

33. The system of claim 29 wherein the control element includes radio buttons.

34. The system of claim 29 wherein the parameter includes a frequency of the pulse.

35. The system of claim 29 wherein the parameter includes a shape of the pulse.

36. The system of claim 29 wherein the parameter includes an amplitude of the pulse.

37. The system of claim 29 wherein the GUI further includes an adjustable control element and an indication element associated with a height of a bottle of irrigation fluid mounted on an IV pole;
the control unit being configured to change the height of the bottle at least in response to adjustments of the control element associated therewith by the user.
